(19) United States
(12) Patent Application Publication
(10) Pub. No.: US 2022/0273504 A1
(43) Pub. Date: Sep. 1, 2022

(54) METHOD OF MANAGING DIFFERENT STAGES IN A WOMEN'S LIFE

(71) Applicant: Katie Rose Hendrix, Loganville, GA (US)

(72) Inventor: Katie Rose Hendrix, Loganville, GA (US)

(21) Appl. No.: 17/733,344

(22) Filed: Apr. 29, 2022

Related U.S. Application Data
(63) Continuation-in-part of application No. 17/189,166, filed on Mar. 1, 2021.

(51) Int. Cl. A61F 13/551; A61K 8/9789; A61K 8/9794; A61Q 19/10; A61K 8/02

(52) U.S. Cl. CPC A61F 13/5519; A61K 8/9789; A61K 8/9794; A61Q 19/10; A61K 8/0208

(57) ABSTRACT

A method for managing different stages in a women's life, the stages related to the reproduction cycle of women. The method includes the steps of recognizing a plurality of stages in a women's life and preparing a kit for each stage. Each kit includes printed publications and healthcare products for use in the management of the stage. The printed publication includes information about the stage and instruction of using healthcare products.

### CROSS-REFERENCE TO RELATED APPLICATIONS

This application is a continuation of U.S. patent application Ser. No. 17/189,166, filed on Mar. 1, 2021, which is hereby incorporated by reference in their entirety.

### FIELD OF INVENTION

The present invention relates to individualized health care, and more particularly, the present invention relates to a method for managing different stages in a women's life.

### BACKGROUND

A Human body changes with time, in particular, the reproductive system affects many stages in human life. However, women's bodies go through major changes throughout their lives compared to men. A women's life can be divided into different stages based on the reproduction cycle and age. In one case, stages in a women's life can include childhood, puberty, reproductive, maturity, menopause, and post-menopause. Different stages affect the body differently and lead to different health concerns in women.

A lot of information about the different stages in the life of women is available in books, magazines, and the internet. However, such information is scattered which the women find difficult to digest. It is a known fact that incomplete knowledge can be harmful. Moreover, it is a time-consuming process to find information from different sources, which may discourage many women from looking for the right information.

Also, different health products are available in the market for different stages in women's life. Such products include tampons, pads, period panties, deodorizers, cleansing wipes, cranberry pills, minerals, and the like. Finding the right products can be troublesome for many women because of the range of products available in the market. Friendly advice may also not help all the time.

Thus, a long-felt need is there for organized healthcare management for different stages in women's life.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a healthcare method for women.

It is another object of the present invention that the method provides for healthcare management of different stages in a women's life.

It is a further object of the present invention is to guide a woman through every cycle of their lives.

In one aspect, disclosed is a method for managing different stages in a women's life. The disclosed method includes the steps of categorizing critical stages in a women's life depending on the age and labeling each stage. Preparing a kit for each stage, wherein the kit contains a printed publication and healthcare products related to the stage. The printed publication includes information about the stage and instructions for using the products.

### DETAILED DESCRIPTION

Subject matter will now be described more fully hereinafter, however, the subject matter may be embodied in a variety of different forms and, therefore, covered or claimed subject matter is intended to be construed as not being limited to any exemplary embodiments set forth herein; exemplary embodiments are provided merely to be illustrative. Likewise, the reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

Disclosed is a method for scientifically managing different stages in a women's life cycle. At first, the method provides for categorizing women's life into precise stages based on the reproductive cycle and age. For example, the girls who have not started their cycle, teenagers who understand the cycle, grown women experiencing a cycle, women who are about to give birth, women who have just given birth (postpartum), and for peri/post-menopausal women. Each of the above stages can be labeled and ordered based on occurrence.

In one exemplary embodiment, the disclosed method provides kits for each of the above stages. Each kit can also be labeled to identify the kit, wherein the label can be descriptively related to the stage. For example, the following kits can be prepared:

CRITICAL BEGINNINGS: This box can be designed for girls above 8 years and can include Sphinx Panties (Period panties), Pads, Cleaning wipes, Tampons, Bath bomb, Vitamin (Iron, cranberry pills), Chamomile tea, and a small zip close bag with essentials (pads, napkins, wipes, & treats, tampon, pain reliever).

TEENS N THINGS: Can be designed for teenagers and includes Loofa, Bath Bombs, Imitation Candlelight, treats, Face Mask, Pads, tampons, Period Panties, Chamomile tea, pain relievers, iron pills, cranberry pills, and cleaning wipes.

ADULTING: For adult, the kit can include wine coolers, treats, nuts, bath bombs, candles, chamomile tea, pain relievers, iron pills, cranberry pills, cleansing wipes, tampons, face masks, period panties, and CBD Treats.

BACK ON THE BOAT I (MATERNITY BOX): This box is designed for mothers-to-be/women who are about to give birth. It prepares them for what they never knew they needed before they need it and includes Nipple Pads, Nipple Butter, Tylenol, Bottom Balm, Aloe Vera Gel, Douche bottle (Portable Bade), postpartum belt, small pillows, and stool softener.

BACK ON THE BOAT II (Postpartum): This box is designed for the women those are having their period again after giving birth and includes Pads, Padsicles, Bath salts, 7 reminders on “good mommy” for the week, vitamins for hair loss, probiotic, 5 ways to bond with your newborn.

Been There. Done That: This box can be designed for peri and post-menopausal women. The items in this box can be designed to balance and relieve women going through this stage of life and can include items from the Adulting Box if desired or needed, essential oils, estrogen, multivitamin, and organic yam chips.

Dot Box To Go: This satchel is an easier way of traveling while on a cycle and can include Tampons, pads (overnight & regular), cleansing wipes, candy, iron pills, cranberry pills, 1 pain reliever, and tea bags.

CATERPILLARS TO BUTTERFLIES: Caterpillars to Butterflies can be a coming-of-age program that can take the sting out of the monthly menstrual cycle. The program can equip each young lady with a box of their own followed with a fun-filled way of understanding how the body works, the changes, and an explanation on why each item is in the box what is designed for, and how it was used. Parents/guardians must attend the session with the caterpillars. Sex education can be discussed only if the parent desires it.

Additionally, the kit may also include a printed publication describing the breakdown of the reproductive system, 1 Gallon a day keeps the cramps away, 1 Date a day for 4 days, how-to session: tampons, pads, period panties, etc., Hygiene, Health & Skin Care, Wait 7 days then “Wade in the Water”.

In one exemplary embodiment, the disclosed method also provides for Virtual Meet-Up of women in the same stages of life. For example, the new mothers can meet up for Storytime, each one teaches one (everyone brings a success story), 15 mins with a Dula, Hygiene, Bringing Sexy back, Bonding with the Baby.

In one exemplary embodiment, the disclosed method can include the step of providing a composition for neutralizing body odors, wherein the kit includes the composition. The composition having Purified Water, Apple Cider Vinegar, Peppermint, Tea Tree, Lavender, Rose Petals, Lemon, Oregano, and Chamomile.

In one exemplary embodiment, the disclosed method also provides for women who have had a hysterectomy. The disclosed method can recognize the need for women who have experienced a hysterectomy in their lifetime. Hysterectomy can be a life-changing procedure and there could be complications after the procedure. The disclosed method can help with the complications. A box can be provided that include treats, primrose oil, bath bomb, 4 pieces of chocolate, 1 candle, 1 Wine Cooler, CDB Gummies, 2 tea bags, a fold-out fan, and yam chips.

In one exemplary embodiment, is disclosed a Prison Box: Complete Commissary (CC) designed for male and female who are currently incarcerated. The box includes Underwear, T-shirts, Razors, Shaving cream, Deodorant, Socks, Brush, Comb, Gloves, Hat, Toothbrush, Toothpaste, Soap, and optionally the Feminine Products (pads, tampons).

In one exemplary embodiment, disclosed is a Box for Thots: S.O.S (Self-Care on Sight) Making the walk of shameless shameful. This box is designed for women who want to be prepared after a hard night of partying and possible intimacy, or a Slumber party. The box can include Panties, Cleansing Wipes, Travel size Toothbrush & toothpaste, Foldout brush.

In one exemplary embodiment, disclosed is a Box designed for men of all ages. It comes with essentials that men normally need and includes Wipes, Cologne, Dark Liquor, Hairbrush, Beard Oil, Condoms, and Candies/Beef Jerky.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for managing stages in a women's life, the method comprising the steps of:
recognizing a plurality of stages in a women's life; and
preparing a kit for each of the plurality of the stages, each kit comprising a plurality of printed publications and a plurality of healthcare products for each of the plurality of the stages.

2. The method according to claim 1, wherein the kit includes a spray composition for neutralizing body odors, the spray composition having the purified water, apple cider vinegar, peppermint, tea tree, lavender, rose petals, lemon, oregano, and chamomile.

3. The method according to claim 1, wherein the kit comprises panties, pads, cleaning wipes, tampons, bath bomb, vitamins, chamomile tea, small zip close bag.

4. The method according to claim 1, wherein the kit comprises loofa, bath bombs, face mask, pads, tampons, period panties, chamomile tea, pain relievers, iron pills, cranberry pills, and cleaning wipes.

5. The method according to claim 1, wherein the kit comprises wine coolers, bath bombs, candles, chamomile tea, pain relievers, iron pills, cranberry pills, cleansing wipes, tampons, face masks, period panties.
